(19) United States
(12) Patent Application Publication
(10) Pub. No.: US 2022/0000911 A1
(43) Pub. Date: Jan. 6, 2022

(54) COMBINATION OF GOLD COMPLEXES WITH OLAPARIB OR OTHER PARP1/2 INHIBITORS FOR USE IN THE TREATMENT OF CANCER RESISTANT TO SAID PARP1/2 INHIBITORS

(71) Applicant: Varsity Pharmaceuticals Limited, Cambridge (GB)

(72) Inventors: James Harrison, Cambridge, Cambridgeshire (GB); Melinda Duer, Cambridge, Cambridgeshire (GB); Uliana Bashtanova, Cambridge, Cambridgeshire (GB)

(21) Appl. No.: 17/293,370

(22) PCT Filed: Sep. 24, 2020
(86) PCT No.: PCT/GB2020/052322

§ 371 Date: May 12, 2021

(51) Int. Cl. A61K 33/242; A61K 31/80; A61K 31/55; A61K 31/502; A61K 31/454; A61K 31/5025; A61K 31/4184; A61K 31/551; A61K 31/519; A61K 31/7135; A61P 35/00

(52) U.S. Cl. CPC A61K 33/242; A61K 31/80; A61K 31/55; A61K 31/502; A61K 31/454; A61P 35/00; A61K 31/4184; A61K 31/551; A61K 31/519; A61K 31/7135; A61K 31/5025

(57) ABSTRACT

The present application is concerned with gold complexes for use in treating cancer which is resistant to a known therapy. In some embodiments, the cancer is resistant to a first PARP inhibitor and/or the gold complex is for use in combination with a second PARP inhibitor.

The invention relates to cancer, and in particular to novel compositions, therapies and methods for treating, preventing or ameliorating cancer.

Cancer cells are characterised by genomic instability, which essentially derives from DNA damage generated by reactive oxygen/nitrogen species (“ROS”), ionising radiation, chemotherapeutic agents and occasional genetic mutations. DNA damage is therefore a direct and indirect target of many cancer treatments.

Eukaryotic cells have developed a sophisticated signaling-transduction mechanism—DNA damage response (“DDR”)—that maintains cell genome integrity. The DDR can detect DNA lesions and repair them, and/or arrest the cell cycle, both temporarily and permanently, and/or promote cell death. Aberrant repair mechanisms and mutations of genes involved in DDR contribute to human cancer onset, development and progression.

Unfortunately, intrinsic or acquired resistance to drug therapies remains an inevitable challenge. Several features such as cell composition of the tumour, tumour microenvironment and drug efficiency lead tumour cells to overwhelm drug therapies through the same mechanisms that healthy cells utilise for surviving under adverse conditions.

Poly-ADP ribose polymerase (PARP) repairs damaged DNA. Several forms of cancer are more dependent on PARP than regular cells, making PARP an attractive target for cancer therapy. As yet, the only FDA-approved class of DDR inhibiting cancer therapies are PARP inhibitors (PARPis). Approved PARPis include olaparib (brand name LYNPARZA (AZ and Merck)), niraparib (brand name ZEJULA (GSK)), rucaparib (brand name RUBRACA (Clovis Oncology)) and talazoparib (brand name TALZENNA (Pfizer)). The first-approved, most-studied and most-used PARPi is olaparib (first FDA approval in 2014).

PARPis inhibit the DNA single-strand break (SSB) repair process—a key component of DDR. Inhibition of SSB can lead to DNA double-strand breaks (DSBs). If SSB repair is inhibited, cancer cells become more reliant on such DSB repair processes. Should such DSB repair processes be inactivated (eg due to genetic mutation) then apoptosis (cell death) can occur.

PARPis are approved for treatment of various classes of ovarian cancer and breast cancer patients (according to the genetic sub-type of the patient and disease progression status). Late stage clinical research is also in progress in prostate cancer, lung cancer and pancreatic cancer, amongst others.

Resistance to PARPi therapy is a significant and growing clinical problem. In particular, recurrent tumours can be more aggressive than the original tumour.

Each approved PARPi has the same mechanism of action regarding DNA single-strand break repair inhibition. This is known as the “PARP-trapping” mechanism whereby the PARPi drug binds to the catalytic domain of PARP1, whilst PARP1 is attached to the DNA lesion. Inhibiting other members of the PARP enzyme family is not required. Due to the existing PARPis having the same mechanism of action and likely to share the same resistance mechanisms, a clinical case showing resistance to one PARPi can already show resistance to all other approved PARPis. Accordingly, PARPi resistance has proven to be a major problem in the clinic.

The present invention arises from the inventors' work in attempting to overcome the problems associated with the prior art.

In accordance with a first aspect there is provided a gold complex for use in treating a cancer which is resistant to a known cancer therapy.

Advantageously, the inventors have found that gold complexes are surprisingly effective at treating cancer cells resistant to known therapies.

In a second aspect, there is provided a method of treating, preventing or ameliorating cancer resistant to a known cancer therapy in a subject, the method comprising administering to a subject in need of such treatment, a therapeutically effective amount of a gold complex.

The gold complex may be a compound of Formula I, Formula II, Formula III, Formula IV or Formula V:

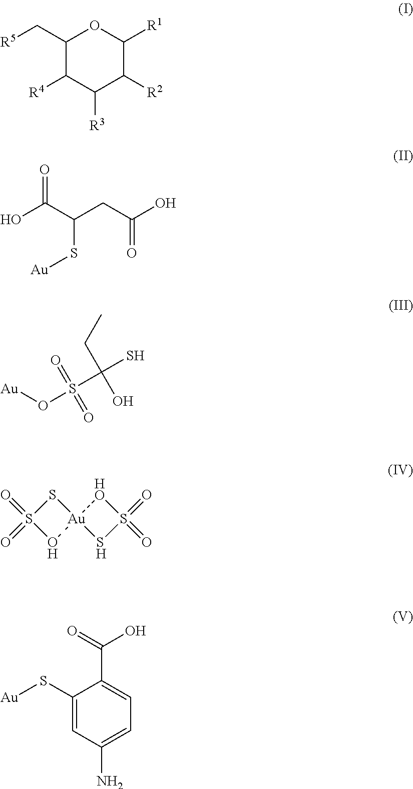

wherein R¹ to R⁵ are each OR⁶, SR⁶, NR⁶R⁷ or SR⁸, and at least one of R¹ to R⁵ is SR⁸;

R⁶ and R⁷ are each independently H, COR⁹, a C₁-C₆ alkyl, a C₂-C₆ alkenyl or a C₂-C₆ alkynyl;

R⁸ is Au or AuPR¹⁰R¹¹R¹²; and

R⁹ to R¹² are each independently H, a C₁-C₆ alkyl, a C₂-C₆ alkenyl or a C₂-C₆ alkynyl;

or a pharmaceutically acceptable salt, solvate, tautomeric form or polymorphic form thereof.

It may be appreciated that atoms in the above compounds may be replaced with isotopes thereof, and the compound will still fall within the scope of the formula. For instance, a hydrogen in one of the above structures could be replaced with a deuterium, and such a compound would fall within the scope of the relevant formula.

Preferably, the gold complex is a compound of Formula (I).

The compound of Formula (I) may be a compound of Formula (Ia):

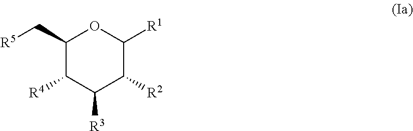

Preferably, one of R¹ to R⁵ is SR⁸.

R¹ to R⁴ are preferably each OR⁶.

R⁶, each time it occurs, is preferably independently H or COR⁹. R⁹, each time it occurs, is preferably C₁-C₃ alkyl, and most preferably methyl. Accordingly, in one embodiment, R⁶, each time it occurs, is COCH₃. In an alternative embodiment, R⁶, each time it occurs, is H.

R⁵ is preferably SR⁸.

In one embodiment R⁸ is AuPR¹⁰R¹¹R¹². R¹⁰ to R¹² are preferably each a C₂-C₄ alkyl, and most preferably are each propyl. Accordingly, R⁸ may be AuP(CH₂CH₃)₃.

In an alternative embodiment, R⁸ is Au.

Accordingly, the gold complex may be auranofin or aurothioglucose or a pharmaceutically acceptable salt, solvate, tautomeric form or polymorphic form thereof.

It may be appreciated that auranofin and aurothioglucose have the following structures:

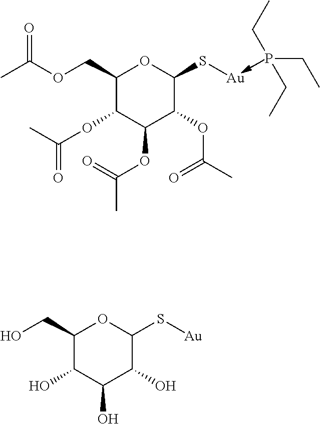

The term “pharmaceutically acceptable salt” may be understood to refer to any salt of a compound provided herein which retains its biological properties and which is not toxic or otherwise undesirable for pharmaceutical use. Such salts may be derived from a variety of organic and inorganic counter-ions well known in the art. Such salts include, but are not limited to: (1) acid addition salts formed with organic or inorganic acids such as hydrochloric, hydrobromic, sulfuric, nitric, phosphoric, sulfamic, acetic, adepic, aspartic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids; or (2) base addition salts formed when an acidic proton present in the parent compound either (a) is replaced by a metal ion, e.g., an alkali metal ion, an alkaline earth ion or an aluminium ion, or alkali metal or alkaline earth metal hydroxides, such as sodium, potassium, calcium, magnesium, aluminium, lithium, zinc, and barium hydroxide, ammonia or (b) coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Pharmaceutically acceptable salts may include, sodium, potassium, calcium, magnesium, ammonium, tetraalkylammonium and the like, and when the compound contains a basic functionality, salts of non-toxic organic or inorganic acids, such as hydrohalides, e.g. hydrochloride, hydrobromide and hydroiodide, carbonate or bicarbonate, sulfate or bisulfate, borate, phosphate, hydrogen phosphate, dihydrogen phosphate, pyroglutamate, saccharate, stearate, sulfamate, nitrate, orotate, oxalate, palmitate, pamoate, acetate, trifluoroacetate, trichloroacetate, propionate, hexanoate, cyclopentylpropionate, glycolate, glutarate, pyruvate, lactate, malonate, succinate, tannate, tartrate, tosylate, sorbate, ascorbate, malate, maleate, fumarate, tartarate, camsylate, citrate, cyclamate, benzoate, isethionate, esylate, formate, 3-(4-hydroxybenzoyl)benzoate, picrate, cinnamate, mandelate, phthalate, laurate, methanesulfonate (mesylate), methylsulphate, naphthylate, 2-napsylate, nicotinate, ethanesulfonate, 1,2-ethane-disulfonate, 2-hydroxyethanesulfonate, benzenesulfonate (besylate), 4-chlorobenzenesulfonate, 2-naphthalenesulfonate, 4-toluenesulfonate, camphorate, camphorsulfonate, 4-methylbicyclo[2.2.2]-oct-2-ene-1-carboxylate, glucoheptonate, 3-phenylpropionate, trimethylacetate, tert-butylacetate, lauryl sulfate, gluceptate, gluconate, glucoronate, hexafluorophosphate, hibenzate, benzoate, glutamate, hydroxynaphthoate, salicylate, stearate, cyclohexylsulfamate, quinate, muconate, xinofoate and the like.

Hemisalts of acids and bases may also be formed, for example, hemisulphate salts.

The term “solvate” may be understood to refer to a compound provided herein or a salt thereof, that further includes a stoichiometric or non-stoichiometric amount of solvent bound by non-covalent intermolecular forces. Where the solvent is water, the solvate is a hydrate.

The cancer may be a solid tumour or solid cancer. The cancer may be blood cancer, bowel cancer, brain cancer, breast cancer, cervical cancer, endometrial cancer, gastric cancer, liver cancer, lung cancer, ovarian cancer, pancreatic cancer, prostate cancer or skin cancer. The blood cancer may be myeloma. The bowel cancer may be colon cancer or rectal cancer. The brain cancer may be a glioma or a glioblastoma. The breast cancer may be a BRCA positive breast cancer. The breast cancer may be a HER2 positive breast cancer or HER2 negative breast cancer. The breast cancer may be triple negative breast cancer. The liver cancer may be hepatocellular carcinoma. The lung cancer may be non-small cell lung cancer or small cell lung cancer. The skin cancer may be a melanoma.

The cancer may be a cancer which is resistant to treatment by a first PARP inhibitor. The patient may have previously been treated with the first PARP inhibitor. The first PARP inhibitor may be a first PARP1 inhibitor. The first PARP1 inhibitor may be aurothiomalate, aurothioglucose (ATG), rucaparib, olaparib, nirparib, talazoparib, veliparib, pamiparib, 2X-121 or auranofin. Preferably, the first PARP1 inhibitor is rucaparib, olaparib, nirparib, talazoparib, veliparib or pamiparib. In some embodiments, the first PARP1 inhibitor is olaparib.

It is known that when a patient suffering from cancer is treated with a PARP inhibitor the cancer may develop resistance to the PARP inhibitor, reducing the effect of subsequent treatment. The inventors have found that cancer cells which are resistant to treatment with a PARP inhibitor (e.g. olaparib) may be treated with the gold complex.

The gold complex may be for use in combination with a second PARP inhibitor. The second PARP inhibitor may be the same or different to the first PARP inhibitor. The second PARP inhibitor may be a second PARP1 inhibitor. The second PARP1 inhibitor may be aurothiomalate, aurothioglucose (ATG), rucaparib, olaparib, nirparib, talazoparib, veliparib, pamiparib, 2X-121 or auranofin. Preferably, the second PARP1 inhibitor is rucaparib, olaparib, nirparib, talazoparib, veliparib or pamiparib. In some embodiments, the second PARP1 inhibitor is olaparib.

Accordingly, the method may comprise administering the gold complex and a second PARP inhibitor to the subject in need thereof.

As described in the examples, the inventors have surprisingly found that the combination of the gold complex and the second PARP inhibitor has a synergistic effect.

The gold complex may be for use before, after or at the same time as the second PARP inhibitor.

Accordingly, the gold complex, and optionally the second PARP inhibitor, may be used in combination with a chemotherapy drug (or a combination of multiple chemotherapy drugs described herein). The chemotherapy drug may comprise bleomycin, capecitabine, carboplatin, cisplatin, cyclophosphamide, dacarbazine, docetaxel, doxorubicin, epirubicin, eribulin, etoposide, 5-fluorouracil, folinic acid, gemcitabine, methotrexate, mustine, oxaliplatin, paclitaxel, prednisolone, procarbazine, vinblastine, vincristine and/or vinorelbine. The gold complex, and optionally the second PARP inhibitor, may be for use before, after or at the same time as the chemotherapy drug. In a preferred embodiment, the gold complex, and optionally the second PARP inhibitor, is for use after the chemotherapy drug.

Alternatively, or additionally, the gold complex, and optionally the second PARP inhibitor, may be used in combination with a drug that damages DNA or which interferes with the DNA damage response process (DDR). Accordingly, the gold complex, and optionally the second PARP inhibitor, may be used in combination with an ATM inhibitor, an ATR inhibitor, a checkpoint inhibitor, a vascular endothelial growth factor (VEGF) inhibitor or a wee1 inhibitor. The checkpoint inhibitor may be a programmed cell death protein 1 (PD-1) inhibitor, a programmed death-ligand 1 (PD-L1) inhibitor or a cytotoxic T-lymphocyte-associated protein 4 (CTLA-4) inhibitor.

Alternatively, or additionally, the gold complex, and optionally the second PARP inhibitor, may be used in combination with ionising radiation that damages DNA.

The gold complex, and optionally the second PARP inhibitor, may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the gold complex, and optionally the second PARP inhibitor, described herein may be used in a number of ways. Compositions comprising the gold complex, and optionally the second PARP inhibitor, may be administered by inhalation (e.g. intranasally). Compositions may also be formulated for topical use. For instance, creams or ointments may be applied to the skin.

The gold complex, and optionally the second PARP inhibitor, according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the gold complex, and optionally the second PARP inhibitor, used according to the invention is required and which would normally require frequent administration (e.g. at least daily injection).

The gold complex, and optionally the second PARP inhibitor, and compositions according to the invention may be administered to a subject by injection into the blood stream or directly into a site requiring treatment, for example into a cancerous tumour or into the blood stream adjacent thereto. Injections may be intravenous (bolus or infusion) or subcutaneous (bolus or infusion), intradermal (bolus or infusion) or intramuscular (bolus or infusion).

In a preferred embodiment, the gold complex, and optionally the second PARP inhibitor, is administered orally. Accordingly, the gold complex, and optionally the second PARP inhibitor, may be contained within a composition that may, for example, be ingested orally in the form of a tablet, capsule or liquid.

It will be appreciated that the amount of the gold complex, and optionally the second PARP inhibitor, that is required is determined by its biological activity and bioavailability, which in turn depends on the mode of administration, the physiochemical properties of the gold complex, and optionally the second PARP inhibitor, and whether it is being used as a monotherapy, or in a combined therapy. The frequency of administration will also be influenced by the half-life of the gold complex, and optionally the second PARP inhibitor, within the subject being treated. Optimal dosages to be administered may be determined by those skilled in the art, and will vary with the particular gold complex, and optionally the particular second PARP inhibitor, the strength of the pharmaceutical composition, the mode of administration, and the advancement of the cancer. Additional factors depending on the particular subject being treated will result in a need to adjust dosages, including subject age, weight, sex, diet, and time of administration.

The gold complex, and optionally the second PARP inhibitor, may be administered before, during or after onset of the cancer to be treated. Daily doses may be given as a single administration. However, preferably, the gold complex, and optionally the second PARP inhibitor, is given two or more times during a day, and most preferably twice a day.

Generally, a daily dose of between 0.01 μg/kg of body weight and 500 mg/kg of body weight of the gold complex, and optionally the second PARP inhibitor, may be used for treating, ameliorating, or preventing cancer. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

A patient receiving treatment may take a first dose upon waking and then a second dose in the evening (if on a two dose regime) or at 3- or 4-hourly intervals thereafter. Alternatively, a slow release device may be used to provide optimal doses of the gold complex, and optionally the second PARP inhibitor, to a patient without the need to administer repeated doses.

Known procedures, such as those conventionally employed by the pharmaceutical industry (e.g. in vivo experimentation, clinical trials, etc.), may be used to form specific formulations comprising the gold complex, and optionally the second PARP inhibitor, according to the invention and precise therapeutic regimes (such as daily doses of the gold complex, and optionally the second PARP inhibitor, and the frequency of administration). The inventors believe that they are the first to describe a pharmaceutical composition for treating cancer, based on the gold complex and the second PARP inhibitor.

Hence, in a third aspect of the invention, there is provided a pharmaceutical composition for treating cancer comprising (i) a gold complex, (ii) a second PARP inhibitor, and (iii) a pharmaceutically acceptable vehicle.

The pharmaceutical composition can be used in the therapeutic amelioration, prevention or treatment in a subject of cancer.

The gold complex and the second PARP inhibitor may be as defined in relation to the first and second aspects.

The invention also provides, in a fourth aspect, a process for making the composition according to the third aspect, the process comprising contacting a therapeutically effective amount of (i) a gold complex, (ii) a second PARP inhibitor and (iii) a pharmaceutically acceptable vehicle.

A “subject” may be a vertebrate or mammal. Most preferably, the subject is a human being.

A “therapeutically effective amount” of the gold complex, and optionally the second PARP inhibitor, is any amount which, when administered to a subject, is the amount of drug that is needed to treat the cancer.

For example, the therapeutically effective amount of the gold complex, and optionally the second PARP inhibitor, used daily may be from about 0.01 mg to about 2,000 mg, and preferably from about 0.1 mg to about 1,000 mg.

It may be appreciated that the exact dosage may depend upon the gold complex which is provided. Furthermore, in embodiments where the gold complex is for use with a PARP inhibitor the dosage of the PARP inhibitor may depend upon the PARP inhibitor which is selected.

For instance, auranofin may be provided as a daily dose of between 0.01 to 100 mg, more preferably between 0.1 and 50 mg or between 0.5 and 25 mg, and most preferably between 1 and 15 mg or between 4 and 10 mg. For instance, auranofin may be administered as a daily dose of 6 mg/day, either as a single daily dose of 6 mg or twice daily at doses of 3 mg. Furthermore, an increase to a daily dose of 9 mg/day after 6 months, which may be provided as three separate doses of 3 mg.

Alternatively, aurothioglucose may be provided as a monthly dosage of between 5 and 1000 mg, more preferably at a monthly dose of between 10 and 500 mg, more preferably a monthly dose between 20 and 200 mg. For instance, aurothioglucose may be administered as an initial 10 mg IM (intramuscular) test dose, and the patient observed for 15-30 minutes for an adverse and/or allergic reaction. The patient may then be given a dose of 25 mg IM one week later and another and dose of 25 mg IM one week after that. The patient may then be maintained of a 50 mg IM once a week until a cumulative dose of 0.8 to 1 G has been reached. If a clinical response has been documented, the dosage may be reduced to a maintenance dosage of 50 mg intramuscularly every three to four weeks. This maintenance dosage may be continued indefinitely based on this patient's response to and tolerance of aurothioglucose.

Desired dosages for PARP inhibitors can vary highly, and depend upon the PARP inhibitor selected. Suitable daily doses will be known to the skilled person.

For instance, olaparib may be administered as a daily dose between 10 and 1,600 mg, more preferably between 100 and 1,200 mg or between 300 and 1,000 mg, and most preferably between 400 and 800 mg. Olaparib may be administered twice daily, e.g. at a dose of 200 mg, 300 mg or 400 mg given twice daily.

Niraparib may be administered as a daily dose between 1 and 1000 mg, more preferably between 50 and 500 mg or between 100 and 400 mg, and most preferably between 230 and 350 mg. Niraparib may be administered once daily, e.g. at a dose of 300 mg.

Finally, talazoparib may be administered at a daily dose of between 0.01 and 10 mg, more preferably between 0.05 and 5 mg or between 0.1 and 2 mg, and most preferably is between 0.25 and 1 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the gold complex, and optionally the second PARP inhibitor) according to the invention. In tablets, the gold complex, and optionally the second PARP inhibitor, may be mixed with a vehicle having the necessary compression properties in suitable proportions and compacted in the shape and size desired. The powders and tablets preferably contain up to 99% of the gold complex, and optionally the second PARP inhibitor. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The gold complex, and optionally the second PARP inhibitor, according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The gold complex, and optionally the second PARP inhibitor, may be prepared as a sterile solid composition that may be dissolved or suspended at the time of administration using sterile water, saline, or other appropriate sterile injectable medium.

The gold complex, and optionally the second PARP inhibitor, and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The gold complex, and optionally the second PARP inhibitor, used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

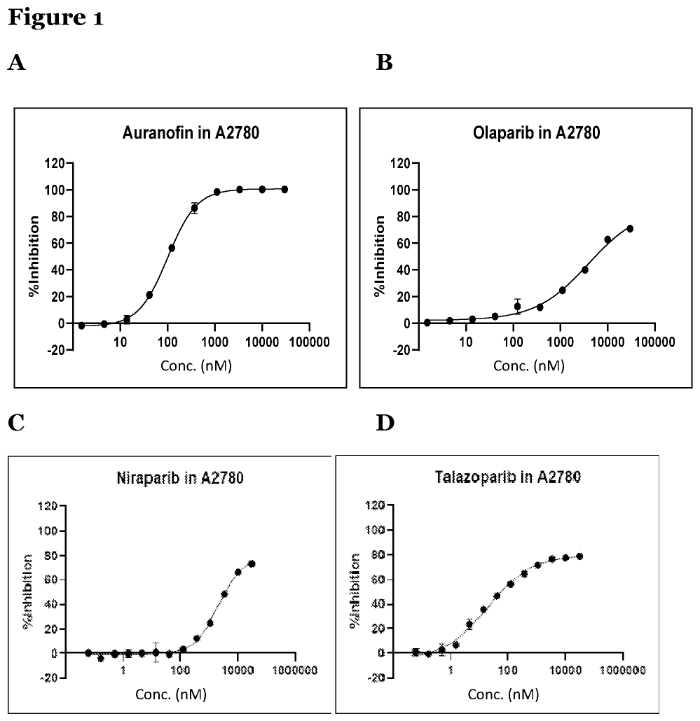

FIG. 1 provides graphs showing the ability of (A) auranofin, (B) olaparib, (C) niraparib and (D) talazoparib to inhibit cell proliferation of wild type A2780 cells at different concentrations;

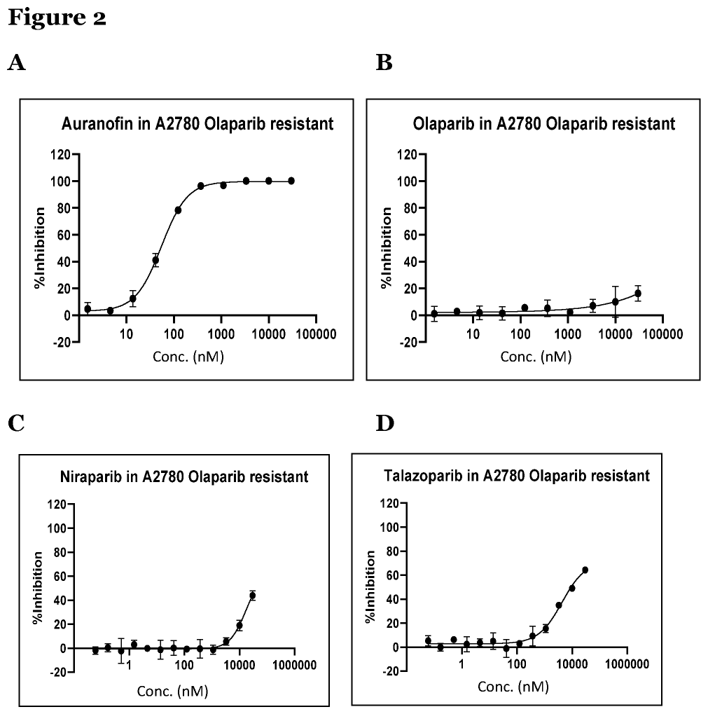

FIG. 2 provides graphs showing the ability of (A) auranofin, (B) olaparib, (C) niraparib and (D) talazoparib to inhibit cell proliferation of olaparib resistant A2780 cells at different concentrations;

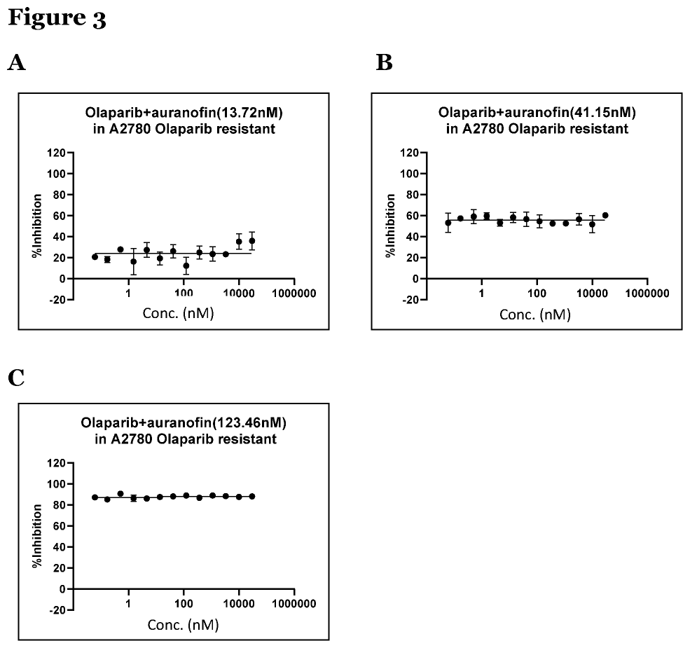

FIG. 3 provides graphs showing the ability of the combination of auranofin and olaparib to inhibit cell proliferation of olaparib resistant A2780 cells at different concentrations;

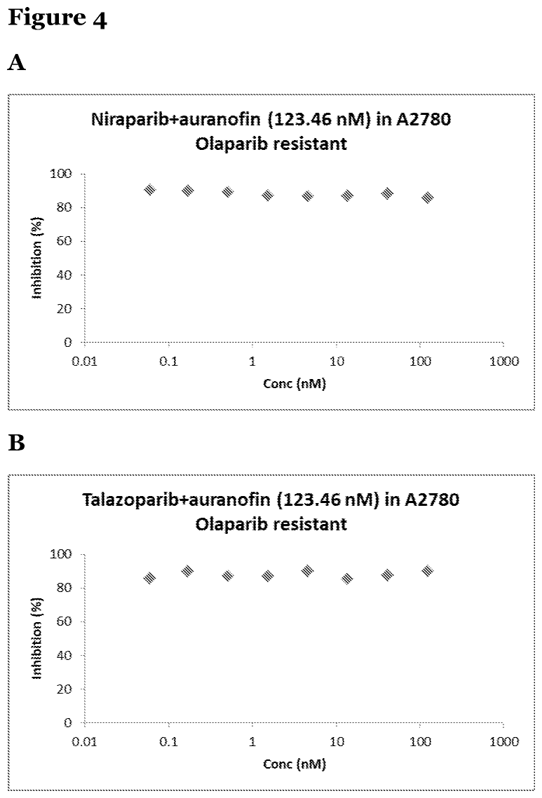

FIG. 4 provides graphs showing the ability of the combination of auranofin and (A) niraparib or (B) talazoparib to inhibit cell proliferation of olaparib resistant A2780 cells at different concentrations;

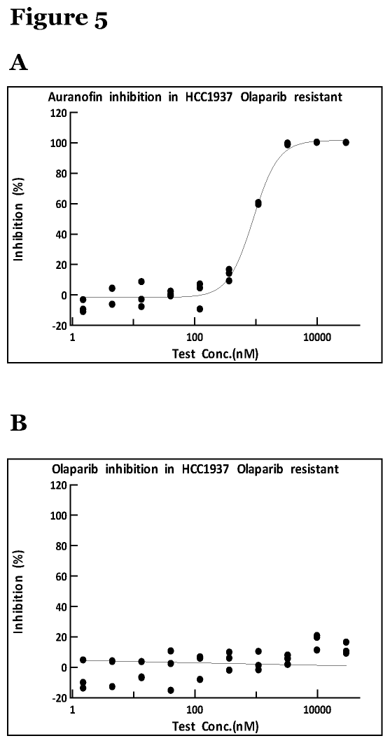

FIG. 5 provides graphs showing the ability of (A) auranofin and (B) olaparib to inhibit cell proliferation of olaparib resistant HCC1937 cells at different concentrations;

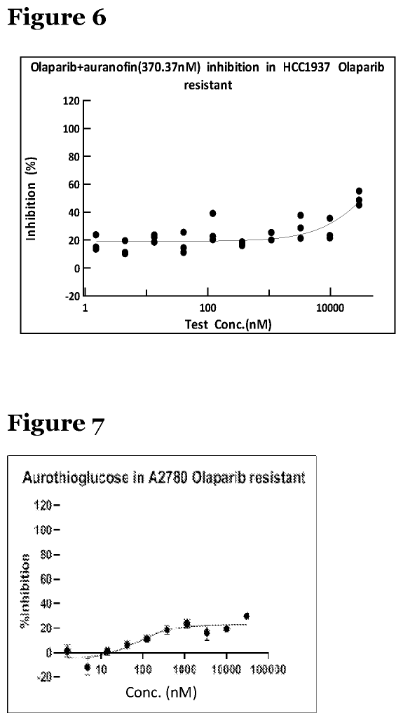

FIG. 6 provides a graph showing the ability of the combination of auranofin and olaparib to inhibit cell proliferation of olaparib resistant HCC1937 cells at different concentrations;

FIG. 7 is a graph showing the ability of aurothioglucose (ATG) to inhibit cell proliferation of olaparib resistant A2780 cells at different concentrations; and

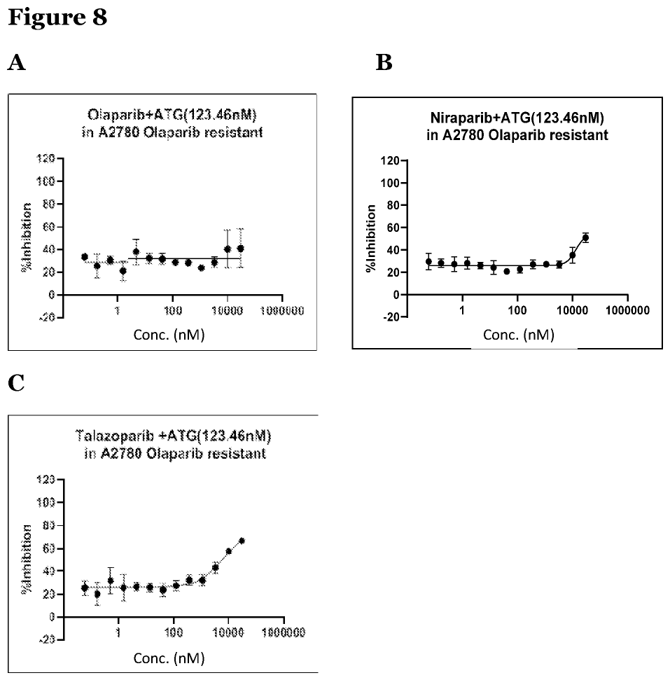

FIG. 8 provides graphs showing the ability of the combination of ATG at a concentration of 123.46 nM in combination with various concentration of (A) olaparib, (B) niraparib and (C) talazoparib to inhibit cell proliferation of olaparib resistant A2780 cells.

### EXAMPLE 1—MEASURING THE ABILITY OF AURANOFIN AND VARIOUS PARP INHIBITORS (PARPIS) TO INHIBIT OLAPARIB RESISTANT A2780 OVARIAN CANCER CELLS

### Methods

### Preparing Cells Resistant to Olaparib

A2780 ovarian cancer cells (wild-type “WT”) were cultured in RPMI 1640 Medium with 10% Fetal Bovine Serum (FBS).

The A2780 cell line is an ovarian cancer cell line that was established from an ovarian endometroid adenocarcinoma tumour. The patient from whom the A2780 cell line was established, did not receive treatment for their tumour before tissue was taken, and so the cell line has not been exposed to any anticancer drugs or chemicals. It is commonly used as a model to observe the effects of, and test the potency of various chemicals, methods of delivery and treatments for ovarian cancer.

To obtain cells resistant to olaparib, the cells were cultured in cell culture medium (RPMI 1640 Medium with 10% FBS) supplemented with 20 μM olaparib for one month in order to generate A2780 cells resistant to olaparib (“R”). After a month the cells were continuously cultured in the cell culture medium (RPMI 1640 Medium with 10% FBS) supplemented with 10 μM olaparib prior to use and for up to two months to maintain the resistance.

### Cell Seeding

The cells were harvested from a flask into cell culture medium (RPMI 1640 Medium with 10% FBS) and then counted. The cells were diluted with culture medium and 40 μL of cell suspension (1000 cells/well) was added into each well of a 384-well cell culture plate. The plate was covered with a lid and left at room temperature without shaking for 30 minutes. The plate was then transferred to an incubator at 37° C. and 5% CO₂ and left overnight.

### Compound Preparation and Treatment

Test compounds were dissolved at a concentration of 30 mM in DMSO to create a stock solution. 45 μL of the stock solution was transferred to a 384 pp-plate. A 3-fold, 10-point dilution was performed by transferring 15 μL of the compound solution into 30 μL DMSO by using TECAN (EVO200) liquid handler. The plate was then spun at room temperature at 1,000 RPM for 1 minute. 40 nL of the diluted compound was then transferred from the compound source plate into the cell plate. The cell plate was then covered with a lid and placed in an incubator at 37° C. and 5% CO₂ and left for 120 hours. 72 hours after treatment with the compound detection was conducted as discussed below.

### Detection

CellTiter Glo® reagents were thawed and equilibrated to room temperature. The cell plate was then removed from the incubator and equilibrated at room temperature for 15 minutes. 30 μL of the CellTiter Glo® reagents were then added into each well (at 1:1 to culture medium). The contents was mixed for 2 minutes on an orbital shaker to induce cell lysis and then the plates were allowed to incubate at room temperature for 30 minutes. Luminescence was then measured on an Envision reader (Perkin Elmer).

### Data Analysis

The inhibition activity was calculated using the following formula:

Inhibition (%)=100×(Lum_{vehicle}−Lumₛₐₘₚₗₑ)/(Lum_{vehicle}−Lum_{blank}),

where Lum_{vehicle} is the luminescence of cells treated with 0.1% DMSO and Lum_{blank} is cells in the culture medium.

The IC₅₀ was calculated by fitting the curve using Xlfit (v5.3.1.3), equation 201:

Y=Bottom+(Top−Bottom)/(1+10\hat{ }((Log IC50−X)*HillSlope))

### Results

The results are shown in Tables 1 to 4 and FIGS. 1 and 2.

TABLE 1
Inhibition of proliferation of wildtype A2780
ovarian cancer cells by auranofin and olaparib

| Test | Mean Inhibition (n = 3)/% | | | |
|---|---|---|---|---|
| Conc./nM | Auranofin | Olaparib | Niraparib | Talazoparib |
| 0.06 | | | 0.39 | 0.69 |
| 0.17 | | | −3.72 | −0.65 |
| 0.51 | | | −0.34 | 2.86 |
| 1.52 | −1.87 | 0.36 | 0.03 | 6.64 |
| 4.57 | −0.74 | 1.77 | 0.18 | 23.37 |
| 13.72 | 2.52 | 2.98 | 0.97 | 35.80 |
| 41.15 | 21.08 | 5.00 | −0.46 | 46.34 |
| 123.46 | 56.32 | 12.51 | 3.18 | 56.34 |
| 370.37 | 86.25 | 11.87 | 12.27 | 64.48 |
| 1,111.11 | 98.39 | 24.63 | 25.03 | 71.38 |
| 3,333.33 | 100.15 | 39.88 | 48.63 | 76.54 |
| 10,000 | 100.17 | 62.69 | 66.23 | 77.63 |
| 30,000 | 100.20 | 70.80 | 73.33 | 78.90 |

TABLE 2
Inhibition of proliferation of olaparib resistant A2780 ovarian
cancer cells by auranofin, olaparib, niraparib and talazoparib

| Test | Mean Inhibition (n = 3)/% | | | |
|---|---|---|---|---|
| Conc./nM | Auranofin | Olaparib | Niraparib | Talazoparib |
| 0.06 | | | −1.98 | 5.28 |
| 0.17 | | | 0.32 | −0.08 |
| 0.51 | | | −2.36 | 6.18 |
| 1.52 | 4.64 | 1.01 | 2.95 | 2.33 |
| 4.57 | 3.42 | 2.74 | −0.19 | 3.63 |
| 13.72 | 12.34 | 1.78 | −1.43 | 4.9 |
| 41.15 | 41.07 | 1.43 | 0.1 | −1.18 |
| 123.46 | 78.31 | 5.53 | −0.8 | 2.95 |
| 370.37 | 96.38 | 5.11 | −0.56 | 9.21 |
| 1,111.11 | 96.82 | 2.24 | −1.37 | 15.45 |
| 3,333.33 | 100.13 | 6.97 | 5.54 | 34.89 |
| 10,000 | 100.19 | 9.9 | 18.95 | 49.04 |
| 30,000 | 100.17 | 16.23 | 43.86 | 64.27 |

TABLE 3
IC₅₀ for auranofin, olaparib, niraparib and talazoparib
in wildtype and olaparib resistance A2780 cells

| | Auranofin | Olaparib | Niraparib | Talazoparib |
|---|---|---|---|---|
| IC₅₀ in wild-type | 101 | 3,889 | 2,087 | 19 |
| A2780 cells/nM | | | | |
| IC₅₀ in olaparib | 55 | >30,000 | 17,272 | 4,314 |
| resistant A2780 | | | | |
| cells/nM | | | | |

TABLE 4
Maximum proliferation inhibition for auranofin, olaparib, niraparib
and talazoparib for concentration ranges between 0.06 nM and
30,000 nM in wildtype and olaparib resistance A2780 cells

| | Auranofin | Olaparib | Niraparib | Talazoparib |
|---|---|---|---|---|
| Maximum | 100 | 72 | 75 | 80 |
| inhibition | | | | |
| observed in | | | | |
| wild-type | | | | |
| A2780 cells/% | | | | |
| Maximum | 100 | 23 | 47 | 66 |
| inhibition | | | | |
| observed in | | | | |
| olaparib | | | | |
| resistant | | | | |
| A2780 cells/% | | | | |

Surprisingly, auranofin appears to be a better inhibitor of proliferation of the olaparib resistant cells than of the wildtype cells, and achieved 100% inhibition for both.

Meanwhile, it is clear from the data provided above that the olaparib resistant cells are also resistant to niraparib and talazoparib. This is due to the same resistance mechanism occurring for all three inhibitors in this case. Accordingly, switching a patient from olaparib monotherapy to niraparib or talazoparib monotherapy would not be successful in this case.

### EXAMPLE 2—MEASURING THE ABILITY OF A COMBINATION OF AURANOFIN AND A PARPI TO INHIBIT THE PROLIFERATION OF OLAPARIB RESISTANT A2780 OVARIAN CANCER CELLS

### Methods

The methods were as described in example 1. In each test, the cells were treated with a combination or auranofin and olaparib. The concentration of auranofin was kept constant while the concentration of olaparib was varied.

All tests were run in triplicate and the mean inhibition calculated.

### Data Analysis

Synergy or antagonism in mixtures may be assessed using the Colby approach. Using this approach calculates an expected result (E) for a mixture of A and B if there is no synergy or antagonism. E may be calculated using the following equation:

E=X+Y−XY/100

Where X is the observed result for compound A and Y is the observed result for compound B. If the observed value is greater than E then this demonstrates synergy.

### Results

The results are shown in Tables 5 and 6 and FIGS. 3 and 4.

TABLE 5
Inhibition of proliferation of olaparib resistant A2780 ovarian
cancer cells by the combination of auranofin and olaparib

| | Olaparib in | | Olaparib in | | Olaparib in | |
|---|---|---|---|---|---|---|
| | combination with | | combination with | | combination with | |
| Test | 13.72 nM auranofin | | 41.15 nM auranofin | | 123.46 nM auranofin | |
| Conc. of | Expected | Observed | Expected | Observed | Expected | Observed |
| Olaparib/nM | Inhibition/% | Inhibition/% | Inhibition/% | Inhibition/% | Inhibition/% | Inhibition/% |
| 0.06 | | 20.68 | | 53.14 | | 87.16 |
| 0.17 | | 18.37 | | 57.27 | | 85.15 |
| 0.51 | | 27.82 | | 59.03 | | 90.67 |
| 1.52 | 13.23 | 16.27 | 41.67 | 59.59 | 78.53 | 86.37 |
| 4.57 | 14.74 | 27.26 | 42.68 | 53.07 | 78.90 | 86.16 |
| 13.72 | 13.90 | 19.23 | 42.12 | 58.18 | 78.70 | 87.48 |
| 41.15 | 13.59 | 26.16 | 41.91 | 56.57 | 78.62 | 88.04 |
| 123.46 | 17.19 | 12.31 | 44.33 | 54.50 | 79.51 | 88.87 |
| 370.37 | 16.82 | 24.90 | 44.08 | 52.41 | 79.42 | 86.8o |
| 1,111.11 | 14.30 | 23.42 | 42.39 | 52.55 | 78.80 | 88.85 |
| 3,333.33 | 18.45 | 23.22 | 45.18 | 56.45 | 79.82 | 88.29 |
| 10,000 | 21.02 | 35.24 | 46.90 | 51.71 | 80.46 | 87.61 |
| 30,000 | 26.57 | 35.97 | 50.63 | 60.26 | 81.83 | 88.08 |

TABLE 6
Inhibition of proliferation of olaparib resistant A2780 ovarian cancer
cells by the combination of auranofin and niraparib or talazoparib

| | Niraparib in | | Talazoparib in | |
|---|---|---|---|---|
| | combination with | | combination with | |
| Test | 123.46 nM auranofin | | 123.46 nM auranofin | |
| Conc. of | Expected | Observed | Expected | Observed |
| PARPi/nM | Inhibition/% | Inhibition/% | Inhibition/% | Inhibition/% |
| 0.06 | 77.88 | 90.25 | 83.59 | 85.86 |
| 0.17 | 78.38 | 89.77 | 78.23 | 90.04 |
| 0.51 | 77.80 | 89.04 | 84.49 | 86.88 |
| 1.52 | 78.95 | 86.82 | 80.64 | 87.09 |
| 4.57 | 78.27 | 86.43 | 81.94 | 89.75 |
| 13.72 | 78.00 | 86.92 | 83.21 | 85.08 |
| 41.15 | 78.33 | 88.11 | 77.13 | 87.26 |
| 123.46 | 78.14 | 85.74 | 81.26 | 89.90 |

The observed proliferation inhibition for the combination of auranofin and a PARPi was consistently higher than the expected values. This indicates that the combination of auranofin and a PARAi exhibits a synergistic effect on the olaparib resistant cells.

The expected values could not be calculated for lower concentrations of olaparib, due to no tests having been conducted to provide the percentage inhibition for olaparib as the sole active agent at these concentrations. However, the observed inhibition is still relatively high, indicating that a synergistic effect is observed down to concentrations of 60 pM olaparib. This conclusion is reinforced by the observed values being significantly higher than the expected values for the lower concentrations of niraparib and talazoparib.

The above results are particularly surprising given that you would not expect cells which are resistant to a PARPi when used alone to display sensitivity to it in combination with a second active agent.

### EXAMPLE 3—MEASURING THE ABILITY OF A COMBINATION OF AURANOFIN AND OLAPARIB TO INHIBIT OLAPARIB RESISTANT HCC1937 TRIPLE NEGATIVE BREAST CANCER CELLS

### Methods

The methods were as described in examples 1 and 2, except the HCC1937 cell line was used.

The HCC1937 cell line was established from a primary breast carcinoma from a 24-year-old patient with a germ-line BRCA1 mutation. It is an example of a triple-negative breast cancer (TNBC) cell line. Breast cancer patients routinely have the expression of estrogen receptor (ER), progesterone receptor (PR), and amplification of HER-2/Neu evaluated. These markers allow classification of breast cancer tumours as hormone receptor positive tumours, HER-2/Neu amplified tumours, and those tumours which do not express ER, PR, and do not have HER-2/Neu amplification. The latter group is referred to as triple-negative breast cancer based on the lack of these three molecular markers. TNBC represents approximately 10-15% of all breast cancers and patients with TNBC have a poor outcome compared to the other subtypes of breast cancer.

### Results

The results are shown in Tables 7 and 8 and FIGS. 5 and 6.

TABLE 7
Inhibition of proliferation of olaparib resistant HCC1937 triple
negative breast cancer cells by auranofin and olaparib

| | |
|---|---|
| Test | Mean Inhibition (n = 3)/% |

| Conc./nM | Auranofin | Olaparib |
|---|---|---|
| 1.52 | −8.11 | −6.55 |
| 4.57 | 0.54 | −1.82 |
| 13.72 | −0.91 | −3.47 |
| 41.15 | 0.49 | −0.93 |
| 123.46 | 0.54 | 1.32 |
| 370.37 | 13.11 | 4.41 |
| 1,111.11 | 59.64 | 3.09 |
| 3,333.33 | 98.95 | 4.90 |
| 10,000 | 100.03 | 17.02 |
| 30,000 | 99.97 | 11.84 |

TABLE 8
Inhibition of proliferation of olaparib resistant HCC1937 triple
negative breast cancer cells by auranofin at a concentration
of 370.37 nM and olaparib at varying concentrations

| Test Conc. of | Expected | Observed |
|---|---|---|
| Olaparib/nM | Inhibition/% | Inhibition/% |
| 1.52 | 7.42 | 17.01 |
| 4.57 | 11.53 | 13.23 |
| 13.72 | 10.09 | 21.04 |
| 41.15 | 12.30 | 16.65 |
| 123.46 | 14.26 | 26.96 |
| 370.37 | 16.94 | 17.08 |
| 1,111.11 | 15.79 | 23.22 |
| 3,333.33 | 17.37 | 28.82 |
| 10,000 | 27.90 | 26.40 |
| 30,000 | 23.40 | 49.22 |

Similar to the results observed in Example 2, a synergistic effect is also observed for the combination of olaparib and auranofin. It is noted that this effect is not observed for the results where olaparib was used at a concentration of 10,000 nM. However, since a synergistic effect is observed at both higher and lower concentrations of olaparib, it is thought that this one result is most likely due to experimental error.

Accordingly, the results show that the combination of auranofin and a PARPi may be used synergistically to inhibit proliferation of multiple cancer cell lines.

### EXAMPLE 4—MEASURING THE ABILITY OF A COMBINATION OF AUROTHIOGLUCOSE AND PARPIS TO INHIBIT PROLIFERATION OF OLAPARIB RESISTANT A2780 OVARIAN CANCER CELLS

### Methods

The methods were as described in examples 1 and 2, except aurothioglucose was used instead of auranofin was used.

### Results

The ability of olaparib, niraparib and talazoparib to inhibit proliferation of olaparib resistant A2780 ovarian cancer cells is provided in table 2 (above). Meanwhile, the ability of aurothioglucose (ATG) to inhibit these cells is provided in Table 9 and FIG. 7.

TABLE 9
Inhibition of proliferation of olaparib resistant
A2780 ovarian cancer cells by aurothioglucose

| | Mean Inhibition |
|---|---|
| Test Conc./nM | (n = 3)/% |
| 1.52 | 1.52 |
| 4.57 | −11.62 |
| 13.72 | 1.10 |
| 41.15 | 6.50 |
| 123.46 | 11.16 |
| 370.37 | 18.52 |
| 1,111.11 | 23.75 |
| 3,333.33 | 16.36 |
| 10,000 | 19.40 |
| 30,000 | 29.94 |

The inhibitor effect of ATG in combination with a PARPi was also investigated and the results are provided in Table 10 and FIG. 8.

TABLE 10
Inhibition of proliferation of olaparib resistant A2780 ovarian cancer cells by aurothioglucose
at a concentration of 123.46 nM in combination with a PARPi at various concentrations

| | Olaparib in | | Niraparib in | | Talazoparib in | |
|---|---|---|---|---|---|---|
| | combination with | | combination with | | combination with | |
| Test | 123.46 nM ATG | | 123.46 nM ATG | | 123.46 nM ATG | |
| Conc. of | Expected | Observed | Expected | Observed | Expected | Observed |
| PARPi/nM | Inhibition/% | Inhibition/% | Inhibition/% | Inhibition/% | Inhibition/% | Inhibition/% |
| 0.06 | | 33.43 | | 29.51 | | 25.33 |
| 0.17 | | 25.52 | | 28.18 | | 20.25 |
| 0.51 | | 30.68 | | 27.23 | | 32.03 |
| 1.52 | 12.06 | 20.97 | 13.78 | 27.95 | 13.23 | 25.49 |
| 4.57 | 13.59 | 37.97 | 10.99 | 25.94 | 14.38 | 26.63 |
| 13.72 | 12.74 | 32.22 | 9.89 | 24.16 | 15.51 | 25.74 |
| 41.15 | 12.43 | 31.69 | 11.25 | 20.61 | 10.11 | 23.68 |
| 123.46 | 16.07 | 28.99 | 10.45 | 22.60 | 13.78 | 27.40 |
| 370.37 | 15.70 | 28.48 | 10.66 | 27.00 | 19.34 | 32.25 |
| 1,111.11 | 13.15 | 23.90 | 9.94 | 27.03 | 24.89 | 32.24 |
| 3,333.33 | 17.35 | 28.82 | 16.08 | 26.67 | 42.16 | 43.05 |
| 10,000 | 19.96 | 40.20 | 28.00 | 35.25 | 54.73 | 57.40 |
| 30,000 | 25.58 | 40.90 | 50.13 | 50.90 | 68.26 | 66.71 |

Again, a clear synergistic effect is observed. This indicates that the synergistic effect is not limited to auranofin, but may be extended to further gold complexes in combination with a PARPi.

1. A gold complex for use in treating a cancer which is resistant to a known cancer therapy.

2. The gold complex for use in accordance with claim 1, wherein the gold complex is a compound of Formula I, Formula II, Formula III, Formula IV or Formula V: 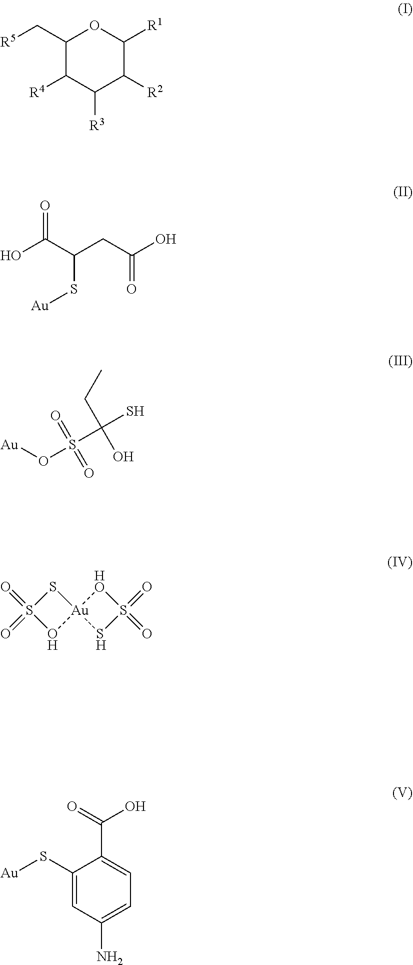
wherein R¹ to R⁵ are each OR⁶, SR⁶, NR⁶R⁷ or SR⁸, and at least one of R¹ to R⁵ is SR⁸;
R⁶ and R⁷ are each independently H, COR⁹, a C₁-C₆ alkyl, a C₁-C₆ alkenyl or a C₂-C₆ alkynyl;
R⁸ is Au or AuPR¹⁰R¹¹R¹²; and
R⁹ to R¹² are each independently H, a C₁-C₆ alkyl, a C₂-C₆ alkenyl or a C₂-C₆ alkynyl;
or a pharmaceutically acceptable salt, solvate, tautomeric form or polymorphic form thereof.

3. The gold complex for use according to claim 2, wherein the gold complex is a compound of Formula (I).

4. The gold complex for use according to claim 3, wherein the compound of Formula (I) is a compound of Formula (Ia):

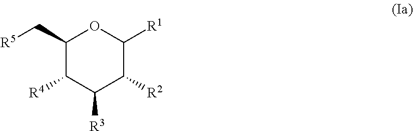

5. The gold complex for use according to claim 3 or claim 4, wherein R¹ to R⁴ are each OR⁶.

6. The gold complex for use according to any one of claims 3 to 5, wherein R⁶, each time it occurs, is independently H or COR⁹ and R⁹, each time it occurs, is a C₁-C₃ alkyl.

7. The gold complex for use according to claim 6, wherein R⁶, each time it occurs, is COCH₃.

8. The gold complex for use according to claim 6, wherein R⁶, each time it occurs, is H.

9. The gold complex for use according to any one of claims 3 to 8, wherein R⁸ is AuPR¹⁰R¹¹R¹² and R¹⁰ to R¹² are each a C₂-C₄ alkyl.

10. The gold complex for use according to any one of claims 3 to 8, wherein R⁸ is Au.

11. The gold complex for use according to claim 1, wherein the gold complex is auranofin or aurothioglucose or a pharmaceutically acceptable salt, solvate, tautomeric form or polymorphic form thereof.

12. The gold complex for use according to any preceding claim, wherein the cancer is a solid tumour or solid cancer.

13. The gold complex for use according to any preceding claim, wherein the cancer is blood cancer, bowel cancer, brain cancer, breast cancer, cervical cancer, endometrial cancer, gastric cancer, liver cancer, lung cancer, ovarian cancer, pancreatic cancer, prostate cancer or skin cancer.

14. The gold complex for use according to any preceding claim, wherein the cancer is a cancer which is resistant to treatment by a first PARP inhibitor.

15. The gold complex for use according to claim 14, wherein a patient suffering from the cancer has previously been treated with the first PARP inhibitor.

16. The gold complex for use according to either claim 14 or claim 15, wherein the first PARP inhibitor is a first PARP1 inhibitor.

17. The gold complex for use according to any preceding claim, wherein the gold complex is for use in combination with a second PARP inhibitor.

18. The gold complex for use according to claim 17, wherein the second PARP inhibitor is a second PARP1 inhibitor.

19. The gold complex for use according to claim 18, wherein the second PARP1 inhibitor is aurothiomalate, aurothioglucose (ATG), rucaparib, olaparib, nirparib, talazoparib, veliparib, pamiparib, 2X-121 or auranofin.

20. The gold complex for use according to claim 19, wherein the second PARP1 inhibitor is rucaparib, olaparib, nirparib, talazoparib, veliparib or pamiparib.

21. A pharmaceutical composition for treating cancer comprising (i) a gold complex, (ii) a second PARP inhibitor, and (iii) a pharmaceutically acceptable vehicle.

22. A process for making the composition of claim 21, the process comprising contacting a therapeutically effective amount of (i) a gold complex, (ii) a second PARP inhibitor and (iii) a pharmaceutically acceptable vehicle.
